SEQUENCE LISTING (95 sequences)

SEQ ID NO: 1 - RNA, 4 nt, Artificial
  Aptamer
accg

SEQ ID NO: 2 - RNA, 4 nt, Artificial
  Aptamer
uccg

SEQ ID NO: 3 - RNA, 7 nt, Artificial
  Aptamer
uuuaccg

SEQ ID NO: 4 - RNA, 7 nt, Artificial
  Aptamer
uucaccg

SEQ ID NO: 5 - RNA, 12 nt, Artificial
  Aptamer
uucaccguaagg

SEQ ID NO: 6 - RNA, 12 nt, Artificial
  Aptamer
aucaccguaagg

SEQ ID NO: 7 - RNA, 12 nt, Artificial
  Aptamer
uuuaccguaagg

SEQ ID NO: 8 - RNA, 12 nt, Artificial
  Aptamer
uuuuccguaagg

SEQ ID NO: 9 - RNA, 12 nt, Artificial
  Aptamer
uuuaccguaagg

SEQ ID NO: 10 - RNA, 12 nt, Artificial
  Aptamer
aucaccguaagg

SEQ ID NO: 11 - RNA, 12 nt, Artificial
  Aptamer
uccaccguagau

SEQ ID NO: 12 - RNA, 12 nt, Artificial
  Aptamer
aucaccguaagg

SEQ ID NO: 13 - RNA, 12 nt, Artificial
  Aptamer
uuuaccguaagg

SEQ ID NO: 14 - RNA, 12 nt, Artificial
  Aptamer
uccaccguaaga

SEQ ID NO: 15 - RNA, 12 nt, Artificial
  Aptamer
uccaccguaaga

SEQ ID NO: 16 - RNA, 12 nt, Artificial
  Aptamer
uuuaccguaagg

SEQ ID NO: 17 - RNA, 12 nt, Artificial
  Aptamer
uuuaccguaagg

SEQ ID NO: 18 - RNA, 12 nt, Artificial
  Aptamer
uuuaccguaagg

SEQ ID NO: 19 - RNA, 12 nt, Artificial
  Aptamer
uccaccguaaga

SEQ ID NO: 20 - RNA, 12 nt, Artificial
  Aptamer
uuuuccguaagg

SEQ ID NO: 21 - RNA, 12 nt, Artificial
  Aptamer
uccaccguaaga

SEQ ID NO: 22 - RNA, 12 nt, Artificial
  Aptamer
uuuaccguaagg

SEQ ID NO: 23 - RNA, 12 nt, Artificial
  Aptamer
aucaccguaagg

SEQ ID NO: 24 - RNA, 70 nt, Artificial
  Aptamer
cucgaucaccguaaggacaucuacguaaguguaaugcggcuuguuuuccccaugcgucugcauaucuguu

SEQ ID NO: 25 - RNA, 67 nt, Artificial
  Aptamer
uuuaccguaaggccugucaucguuugacagcggcuuguugacccuuccacuaugugugccuguaaug

SEQ ID NO: 26 - RNA, 72 nt, Artificial
  Aptamer
acuucgucuugcagcgcggcuugucucuucccacauccguucuaucgguaugacucuuuuuccguaagguca

SEQ ID NO: 27 - RNA, 66 nt, Artificial
  Aptamer
uuuaccguaaggccugucuucguuugacagcggcuuguugacccucacacuuuguaccucugccug

SEQ ID NO: 28 - RNA, 69 nt, Artificial
  Aptamer
cucgaucaccguaaggacaucuacauaaguguaaugcggcuuguuuuccccaugcaucugcauaucugu

SEQ ID NO: 29 - RNA, 66 nt, Artificial
  Aptamer
uguccaccguagauuguaaacuaucgcguaaagcgaaguuuauguggcuuguuuucccacgccuug

SEQ ID NO: 30 - RNA, 69 nt, Artificial
  Aptamer
cucgaucaccguaaggacauuuacguaaguguaaugcggcuuguuuuccccaugcgucugcauaucugu

SEQ ID NO: 31 - RNA, 67 nt, Artificial
  Aptamer
uuuaccguaaggccugucuucguuugacagcggcuuguugacccucacacuuuguaccugcugccaa

SEQ ID NO: 32 - RNA, 68 nt, Artificial
  Aptamer
uccaccguaagauuguaaacuaucggguaaagacgaaguuuauguggcuuguuucccaccgccuugcc

SEQ ID NO: 33 - RNA, 69 nt, Artificial
  Aptamer
uguccaccguaagauuguaaacuaucguaaagacgaaguuuauguggcuuguuuucccaccgccuugcc

SEQ ID NO: 34 - RNA, 67 nt, Artificial; identical to SEQ ID NO: 25
  Aptamer

SEQ ID NO: 35 - RNA, 66 nt, Artificial
  Aptamer
uuuaccguaaggccugucuucguuugacagcggcuuguugacccacacacuuugucccggcugcag

SEQ ID NO: 36 - RNA, 69 nt, Artificial
  Aptamer
uuuaccguaaggccugucuucuuuugacagcggcuuguugacccucacgcuuugucccugcuguaccug

SEQ ID NO: 37 - RNA, 68 nt, Artificial
  Aptamer
uccaccguaagauuguaaacuaucgcguaaaagacgaaguuuauguggcuuguuuucccaccgccuug

SEQ ID NO: 38 - RNA, 68 nt, Artificial
  Aptamer
acuucgucuugcagcgcggcuugucuucccacauccguucuaucgguaugacuuuuuccguaagguca

SEQ ID NO: 39 - RNA, 68 nt, Artificial
  Aptamer
uccaccguaagauuguaaacuaucgcguaaagacgaaguuuauguggcuuguuucccaccaccuugcg

SEQ ID NO: 40 - RNA, 67 nt, Artificial
  Aptamer
uuuaccguaaggccugucuucguuugacagcggcuuguugacccucacgcuuugucccaugcccguc

SEQ ID NO: 41 - RNA, 70 nt, Artificial
  Aptamer
cucgaucaccguaaggacauuuacguaaguguaaugcggcuuguuuuccccaugcguuugcauaucugug

SEQ ID NO: 42 - RNA, 40 nt, Artificial
  Aptamer
acguugaguacaaccccaccccguucacgguagcccugua

SEQ ID NO: 43 - RNA, 39 nt, Artificial
  Aptamer
gcuacaguacaacgggccguguggaauacaccgacaagg

SEQ ID NO: 44 - RNA, 40 nt, Artificial
  Aptamer
uccaccgaucuagaugauccaggcacccgaccaccaccuc

SEQ ID NO: 45 - RNA, 37 nt, Artificial
  Aptamer
gcuuguggaccaagaagcaaccagucaccguugcccc

SEQ ID NO: 46 - RNA, 40 nt, Artificial
  Aptamer
caacaguccuguguccguugaauccucuagauccagggug

SEQ ID NO: 47 - RNA, 39 nt, Artificial
  Aptamer
ggaccccccacagcaaguuugccggcgaccgcguucuug

SEQ ID NO: 48 - RNA, 40 nt, Artificial
  Aptamer
cuugccacuccugucuagcuggcguagaccgcgcagaaag

SEQ ID NO: 49 - RNA, 40 nt, Artificial
  Aptamer
gcuaguagccucagcagcauaguuucgccgcuaugcagua

SEQ ID NO: 50 - RNA, 40 nt, Artificial
  Aptamer
uagcauaauguggagcguugaccggaccucuccagucgua

SEQ ID NO: 51 - RNA, 38 nt, Artificial
  Aptamer
uggacuacgcacccgcuaguccguccaagaacugugcg

SEQ ID NO: 52 - RNA, 40 nt, Artificial
  Aptamer
uucuguuccgaccaauugaauagucaccgugaugauuuga

SEQ ID NO: 53 - RNA, 39 nt, Artificial
  Aptamer
gaugccagcgcgcauucuucaccgaaguacguauccacg

SEQ ID NO: 54 - RNA, 39 nt, Artificial
  Aptamer
uucgccgcugcacucucgcagcacuggucgggauguguc

SEQ ID NO: 55 - RNA, 6 nt, Artificial
  Aptamer
gcugaa

SEQ ID NO: 56 - RNA, 40 nt, Artificial
  Aptamer
aguaggaauacccccauccaaagcucgcuaggcugaacac

SEQ ID NO: 57 - RNA, 40 nt, Artificial
  Aptamer
gacggcccgagauugcagaaaaacgcgcccacgugucaga

SEQ ID NO: 58 - RNA, 40 nt, Artificial
  Aptamer
ucccuagcugacgauggaucuuggaucacauaggcugcgc

SEQ ID NO: 59 - RNA, 38 nt, Artificial
  Aptamer
ggacaugccggucuugagcggaggugaaccguaccacg

SEQ ID NO: 60 - RNA, 35 nt, Artificial
  Aptamer
aacacgccucaggacgccaggugaacccucgaacc

SEQ ID NO: 61 - RNA, 39 nt, Artificial
  Aptamer
aacgcugaaucccccggucauagaacuuugauaguacag

SEQ ID NO: 62 - RNA, 40 nt, Artificial
  Aptamer
uacugaaugaucuccacccgccggaaugcguauagucccu

SEQ ID NO: 63 - RNA, 40 nt, Artificial
  Aptamer
gcuggggaaagcagguccguucccaccgccugaagcuuug

SEQ ID NO: 64 - RNA, 40 nt, Artificial
  Aptamer
ccuccugacacaaccacccaaccaccuucuugaaacauuu

SEQ ID NO: 65 - RNA, 41 nt, Artificial
  Aptamer
acaaccuugauugcuugaaaccucuaacccgaggcucugua

SEQ ID NO: 66 - RNA, 5 nt, Artificial
  Aptamer
gaaag

SEQ ID NO: 67 - RNA, 34 nt, Artificial
  Aptamer
cgaacguggacgaagggcgguuugugagugcuua

SEQ ID NO: 68 - RNA, 41 nt, Artificial
  Aptamer
cugacugcgucucuauaugacauaggcgaugagaaagcaga

SEQ ID NO: 69 - RNA, 41 nt, Artificial
  Aptamer
ggaacagacgucaucuguggcacguccgcugcuagcagaga

SEQ ID NO: 70 - RNA, 41 nt, Artificial
  Aptamer
gacacaagcuggaccacgucaagcguuuugugaaagcaggu

SEQ ID NO: 71 - RNA, 41 nt, Artificial
  Aptamer
uggcaucuugugcaugacaacagagggugaaaccaacgggu

SEQ ID NO: 72 - RNA, 38 nt, Artificial
  Aptamer
aaacuugccuugguuuauaacguaacaauacagaacga

SEQ ID NO: 73 - RNA, 38 nt, Artificial
  Aptamer
agaaucuaagacgugaaaaugguaagacauucucuacc

SEQ ID NO: 74 - RNA, 40 nt, Artificial
  Aptamer
aggugugcgcagacgaauaggguugugcgaaagucuagca

SEQ ID NO: 75 - RNA, 40 nt, Artificial
  Aptamer
uuuagaguugaaaugcguaaugguuaaaugauccauucug

SEQ ID NO: 76 - RNA, 40 nt, Artificial
  Aptamer
aacaaugcgaggguaaaagcauguucuaaccagggaggga

SEQ ID NO: 77 - RNA, 36 nt, Artificial
  Aptamer
auggaagcccuugauucuacggaucuagcgagauuu

SEQ ID NO: 78 - RNA, 39 nt, Artificial
  Aptamer
ccuguaagggcgaaacuaagcgagaaaucauuaggauga

SEQ ID NO: 79 - RNA, 45 nt, Artificial
  Aptamer
cucaaugcauacgcuggucaacgggacgauuagugacaaggccgc

SEQ ID NO: 80 - RNA, 39 nt, Artificial
  Aptamer
aauaaguggcaaguagccuagagauuagaagaccucaac

SEQ ID NO: 81 - RNA, 41 nt, Artificial
  Aptamer
aauugacgagcuggugggagauagucucagguaucuugugc

SEQ ID NO: 82 - RNA, 38 nt, Artificial
  Aptamer
gguggacaguaacuccuuagaugcgguagauucguagc

SEQ ID NO: 83 - RNA, 40 nt, Artificial
  Aptamer
uacgcgcuuaugauaaaggguuagaaggacgagcgucgca

SEQ ID NO: 84 - RNA, 39 nt, Artificial
  Aptamer
cacaugcagaguaguguaagguaacacccagguuuuuug

SEQ ID NO: 85 - RNA, 41 nt, Artificial
  Aptamer
ccggggcgcaggugucccugacgaugaucaauuucggguga

SEQ ID NO: 86 - RNA, 39 nt, Artificial
  Aptamer
gacgccuuuaugaaugaccagggaaguugucagaagagg

SEQ ID NO: 87 - RNA, 38 nt, Artificial
  Aptamer
gucacuuucugaaugggagauaucuucgauaugguaau

SEQ ID NO: 88 - RNA, 6 nt, Artificial
  Aptamer
guuaau

SEQ ID NO: 89 - RNA, 41 nt, Artificial
  Aptamer
gaaggcgaaaggcacaaagaucugaugaaguuaauggauca

SEQ ID NO: 90 - RNA, 35 nt, Artificial
  Aptamer
guuaaucgcugaauauucgaagugcuuuccgugau

SEQ ID NO: 91 - RNA, 40 nt, Artificial
  Aptamer
ugggcuuagguguuaagucgaugacuguucauucucggua

SEQ ID NO: 92 - RNA, 40 nt, Artificial
  Aptamer
acgugagcgagcaauaaaaguccccuggggcggaguuaaa

SEQ ID NO: 93 - RNA, 39 nt, Artificial
  Aptamer
gggagagucuacggauccuagaaaaagcaggacguuauu

SEQ ID NO: 94 - RNA, 42 nt, Artificial
  Aptamer
caaaggggagccacggggcgacguguaauccucuauucagca

SEQ ID NO: 95 - RNA, 40 nt, Artificial
  Aptamer
aaugaaggcaauucuuuaacguuaauaggaaggggguaaa

(19) United States
(12) Patent Application Publication
(10) Pub. No.: US 2016/0193202 A1
(43) Pub. Date: Jul. 7, 2016

(54) THERAPEUTIC TREATMENT FOR DRUG POISONING AND ADDICTION

(71) Applicants: ADISPELL, INC., Rochester, NY (US); CORNELL UNIVERSITY, Ithaca, NY (US)

(72) Inventors: Stephen H. CURRY, Victor, NY (US); George P. HESS, Ithaca, NY (US)

(21) Appl. No.: 14/911,942

(22) PCT Filed: Aug. 12, 2014
(86) PCT No.: PCT/US2014/050713

§ 371 Date: Feb. 12, 2016

Related U.S. Application Data
(60) Provisional application No. 61/864,934, filed on Aug. 12, 2013.

(51) Int. Cl. A61K 31/46; A61K 31/5386; C12N 15/115

(52) U.S. Cl. CPC A61K 31/46; C12N 15/115; A61K 31/5386; C12N 2320/30

(57) ABSTRACT

The present invention relates to methods of treating or preventing drug poisoning and drug addiction in a subject. These methods involve administering to a subject in need of said treatment or prevention a ligand which binds to a regulatory site on the nicotinic acetylcholine receptor.

This application claims the benefit of U.S. Provisional Patent Application Ser. No. 61/864,934, filed Aug. 12, 2013, which is hereby incorporated by reference in its entirety.

### FIELD OF THE INVENTION

The present invention relates to methods for therapeutically treating and/or preventing drug addiction and poisoning in a subject.

### BACKGROUND OF THE INVENTION

Drug addiction is a long-standing societal problem that often has an effect on individuals, family members, and society. This addiction is mostly characterized by an intense and uncontrollable craving for the drug, along with compulsive drug seeking and use that continues, at times, in the face of devastating consequences. While the path to drug addiction begins with the voluntary act of taking drugs, over time a person's ability to choose not to take drugs becomes compromised, thus seeking and consuming the drug becomes compulsive. This behavior is a result of the effects of prolonged drug exposure on brain functioning. Addiction is a brain disease that affects multiple brain circuits, including those involved in reward and motivation, learning and memory, and inhibitory control over behavior.

The National Survey on Drug Use and Health by SAMHSA (2007) reported that 23.2 million people (9.4 percent of the U.S. population) aged 12 or older needed treatment for an illicit drug or alcohol use problem.

Current drug treatment programs typically incorporate multiple components, each directed to a particular aspect of the illness and its consequences. Addiction treatment must help an individual to stop using drugs, maintain a drug-free lifestyle, and achieve productive function at work and in society. Addiction is typically a chronic disease; people cannot simply stop using drugs for a time and be cured. Most patients require long-term or repeated episodes of care to achieve the ultimate goal of sustained abstinence and recovery of their lives.

Scientific research has shown that treatment can help drug-addicted patients to stop using drugs, avoid relapse, and successfully recover their lives. Based on this research, the key principles of effective treatment that are most relevant to this invention are: (i) addiction is a complex but treatable disease that affects brain function and behavior; (ii) no single treatment is appropriate for everyone; (iii) medication is an important element of treatment for many patients, especially when combined with counseling and other behavioral therapies, (iv) medically assisted detoxification is only the first stage of addiction treatment and by itself does little to change long-term drug abuse.

Medication and behavioral therapy, especially when combined, are important elements of an overall therapeutic process that most often begins with detoxification, followed by treatment and relapse prevention. Easing withdrawal symptoms can be important with the initiation of treatment. Preventing relapse is necessary for maintaining the effects of withdrawal. A continuum of care that includes a customized treatment regimen—addressing all aspects of an individual's life, including medical and mental health services—and follow-up options (e.g., community—or family-based support systems) can be crucial to success in achieving and maintaining a drug-free lifestyle.

Medications are often used to help with different aspects of the treatment process. For example, medications can be given to offer help in suppressing withdrawal symptoms during detoxification. Medications are used during treatment to help reestablish normal brain function, to prevent relapse, and to diminish cravings. Currently, there are medications for opioids (heroin, morphine), tobacco (nicotine), and alcohol addiction. Under development are others targeted to treat for stimulants (cocaine, methamphetamine) and cannabis (marijuana) addiction. These treatments must be used with behavioral therapy.

Medications administered for the treatment of opiate addiction include methadone, buprenorphine and naltrexone. Acting on the same targets in the brain as heroin and morphine, methadone and buprenorphine suppress withdrawal symptoms and relieve cravings. Naltrexone works by blocking the effects of heroin or other opioids at their receptor sites and should be used only in patients who have been detoxified.

Drug poisoning or toxicity is a different state where an individual may have ingested more of a particular drug than the body can properly process due to illicit ingestion, a therapeutic error, or a suicide attempt. Most life-threatening cases of intoxication do not have a pharmacological treatment and can result in death. A count of 36,500 U.S. deaths due to drug intoxication was registered in 2008, nearly as many as caused by automobile related deaths that year. An indication of the danger of drug abuse is that the number of emergency room visits for drug abuse has risen to 2,070,440 per year.

Phencyclidine (PCP) use and addiction with higher doses, can lead to a wide range of physical effects (e.g., at times increased blood pressure, at times lower blood pressure) and a number of unpleasant behaviors (e.g., at times drowsiness, at times agitation) and results. PCP is often synthesized and its effects on an individual can be unpredictable, e.g., at times being a stimulant, at other times a depressant, and often a hallucinogenic. Use of the drug has been known to cause violent and suicidal behavior, as well as the possibility of seizures, coma, and death with higher consumption. PCP has been known to cause delusions, have psychological consequences, promote risky behavior, with each of these outcomes, along with poisoning, being a potential cause of death.

Long term use can cause memory loss, and difficulty with speech and thought, as well as depression. A recent survey concluded that at least 6 million Americans age 12 and older have used PCP at some time in their life.

According to the National Institute for Drug Abuse, nicotine is considered one of the most widely abused substances. Their 2007 survey estimated that 70.9 million Americans use tobacco products, a primary source of nicotine. Nicotine addiction has many characteristics that are similar to other drug addictions.

A variety of formulations of nicotine replacement therapies now exist—including patches, sprays, inhalers, gums, and lozenges. In addition, two prescription medications—bupropion and varenicline—are FDA—approved for tobacco addiction. They have different mechanisms of action in the brain and often must work in conjunction with behavior modification treatments.

Despite the staggering statistics on drug addiction, specific and effective therapeutic treatments for many drugs of abuse of are lacking The present invention is directed at overcoming these and other deficiencies in the art.

### SUMMARY OF THE INVENTION

The present invention is directed to a method of preventing and/or treating drug poisoning or drug addiction in a subject. This method involves selecting a subject having or at risk of having drug poisoning or a drug addiction and administering to the subject a ligand that binds to a regulatory site on nicotinic acetylcholine receptors (nAChRs) under conditions effective to treat or prevent drug poisoning or drug addiction in the subject.

As described herein, applicants have discovered highly specific and safe alleviators of the effects of phencyclidine and other related compounds. Most treatments for drug addiction and/or poisoning are no more than palliative, damping down the toxic effects of the drug or providing relief during withdrawal. The treatments can themselves be non-specific and have side effects. However, because of the specificity of the compounds of the present invention, toxic and alleviatory compounds can be explored in pairs, such that problems of the individual toxic drugs will be relieved by specifically-designed alleviatory complementary compounds with a high level of precision.

### BRIEF DESCRIPTION OF THE DRAWINGS

FIGS. 1A-1C are Morris water maze traces of three individual rats following vehicle treatment (FIG. 1A), a combined dose of EME (10 mg/kg) and scopolamine (1 mg/kg) (FIG. 1B), and a single dose of scopolamine (1 mg/kg) (FIG. 1C). The target platform was located in the lower left quadrant of the water bath.

FIG. 2 shows time spent in the area previously occupied by the platform in seconds (y-axis) in the Morris water maze test for rats administered vehicle (1), EME alone (2), scopolamine alone (3), and the EME in combination with scopolamine (4).

FIG. 3 is a graph showing brain (ng/g) and plasma (ng/ml) concentrations of ecgonine methyl ester (“EME” or “E compound”) in rats following intraperitoneal administration of a 10 mg/kg dose at 0, 1, 4, 8, and 24 hours.

FIGS. 4A-4D shows the effects on BC₃H1 nicotinic acetylcholine receptor (“nAChR”) currents of a single-cloned Class 1 or Class 2 RNA aptamer or cocaine in the presence of carbamoylcholine (adapted from Ulrich et al., “In Vitro Selection of RNA Molecules that Displace Cocaine from the Membrane-Bound Nicotinic Acetylcholine Receptor,” Proc. Nat. Acad. Sci. 95: 14051-14056 (1998), which is hereby incorporated by reference in its entirety). FIG. 4A shows results of a control experiment. FIG. 4B shows results of an aptamer with no effect on unimpaired carbamoylcholine. FIG. 4C presents the same condition as FIG. 4A, but with the Class 1 compound cocaine present. FIG. 4D shows results of same condition as in FIG. 4A, but with a Class 1 aptamer present.

FIG. 5 shows electrophysiological data showing a Class 2 aptamer alleviating the effect of a Class 1 compound (cocaine) (Hess et al., “Mechanism-Based Discovery of Ligands that Counteract Inhibition of the Nicotinic Acetylcholine Receptor by Cocaine and MK-801,” Proc. Nat. Acad. Sci. 97(25): 13895-13900 (2000), which is hereby incorporated by reference in its entirety).

FIG. 6 shows the alleviation by ecgonine methyl ester (“EME”) of cocaine inhibition of the nAChR. The cells were preincubated with 200-μM cocaine for 50 ms before a solution of carbamoylcholine with or without the other ligands, flowed over the cell (Chen et al., “Mechanism-Based Discovery of Small Molecules that Prevent Noncompetitive Inhibition by Cocaine and MK-801 Mediated by Two Different Sites on the Nicotinic Acetylcholine Receptor,” Biochemistry 43:10149-10156 (2004), which is hereby incorporated by reference in its entirety).

### DETAILED DESCRIPTION OF THE INVENTION

The present invention is directed to a method of preventing and/or treating drug poisoning or drug addiction in a subject. This method involves selecting a subject having or at risk of having drug poisoning or a drug addiction and administering to the subject a ligand that binds to a regulatory site on nicotinic acetylcholine receptors under conditions effective to treat or prevent drug poisoning or drug addiction in the subject

In accordance with present invention, the drug poisoning or a drug addiction in a subject can be caused by any drug, including, without limitation, drugs of abuse, such as phencyclidine (PCP), marijuana, cocaine, nicotine and alcohol, and, in particular, centrally and peripherally acting anticholinergic drugs such as scopolamine.

As used herein “drug addiction” is considered synonymous with a dependence on a drug or a medication. However, historically there has been considered to be a distinction between addiction and dependence, with “addiction” being used to describe a situation in which the body has become physiologically and/or biochemically adapted to the presence of the drug or medication, such that when the drug or medication treatment is stopped physiological and/or biochemical phenomena occur that are unpleasant or even life-threatening. “Dependence” is a term used historically to describe a less severe form of continued need for a drug or medication, with more in common with pursuit of a habit than with organic adaptation, so that withdrawal of the drug or medicine may be disruptive but not biologically unpleasant. Some authorities consider this distinction to be artificial. Addiction and withdrawal effects vary widely with different drugs. There is almost always a craving for more doses with an addictive drug. Addiction can involve lack of control of drug use and continued use even with the knowledge that it is harmful. This is certainly true with phencyclidine, LSD, and heroin addiction. Addiction of this kind cannot be reversed without the help of competent professionals and effective therapy.

Treatment of drug addiction can involve psychotherapy, supportive medication, and specific antidotes to the drugs causing the addiction. For example, there are receptor antagonists of heroin that prevent the euphoria caused by the heroin without stimulating the receptors in the way heroin does. Use of such antidotes requires concomitant medication to reverse the withdrawal effects, which are very severe, such as, in the case of heroin, painful gastrointestinal effects. As a further example, in the case of alcohol addiction, drugs can be used to prevent the ongoing metabolism of the acetaldehyde formed from the alcohol, in the liver, to acetic acid. Acetic acid is without dramatic pharmacological effects, but acetaldehyde causes a severe sickness syndrome, such that the alcoholic individual stops drinking because of the fear of the acetaldehyde effect that will occur upon consumption.

It will be understood that there is a major medical need for drug treatments that reverse the effects of addictive drugs such as phencyclidine. The technology described herein provides a mechanism for achieving such a result with phencyclidine and other drugs of addiction.

In contrast to drug addiction, drug poisoning is usually associated with accidental, suicide-related, or malicious high exposure to drugs that are essentially benign when used at lower doses. This applies in the cases of both therapeutic agents and drugs of abuse. Basically, a poisoning occurs when a person's exposure to a natural or manmade substance has an undesirable effect. Drug poisoning often occurs with illegal, prescription, or over-the-counter drugs. For example, poisoning with prescription opioid painkillers, such as Oxycontin and Vicodin, has reached epidemic proportions in the USA in recent years; deaths from poisoning by drugs of abuse have also increased ten-fold in the last ten years. There are many other examples of common poisonous agents such as benzodiazepine drugs, cyanide, and poisonous plants such as belladonna (nightshade) and poison ivy.

Specific antidotes for acute poisoning are very valuable in emergency departments of hospitals. For example, specific antidotes for belladonna poisoning works by inducing opposite effects via autonomic nervous system based mechanisms. Morphine overdose can be reversed by using morphine receptor antagonist compounds, to block morphine's effects at its receptor. It is readily appreciated that specific antidotes to compounds such as phencyclidine would also be invaluable.

The present invention permits both desirable and undesirable effects to be induced at receptor sites in the brain by various compounds related in pairs by virtue of their respective poison and antidote properties. This includes the discovery that the effects of phencyclidine and related compounds can be alleviated by appropriately chosen “paired” compounds of the present invention as described herein.

In accordance with the methods of the present invention, addiction and poisoning by drugs of abuse in a subject can be treated or prevented by administering to the subject a ligand that binds to nicotinic acetylcholine receptors and treats addiction and poisoning symptoms in the subject. This binding occurs at a binding site distinct from that at which acetylcholine binds. Laboratory work has demonstrated the existence of a regulatory site of the nicotinic acetylcholine receptors that is distinct from the binding site of the natural ligand acetylcholine. As used herein, the term “ligand” includes, but is not limited to, small organic molecules, aptamers, and other compounds that similarly bind to this regulatory site on the nicotinic acetylcholine receptors and induce an allosteric change in the receptors in the presence of the abused drug, thereby changing the channel opening equilibrium of the receptor to enhance the flow of inorganic cations through the receptor channel. The characteristics and binding function of this regulatory site on the nicotinic acetylcholine receptors have previously been described (see e.g., Hess et al., “Mechanism-Based Discovery of Ligands that Counteract Inhibition of the Nicotinic Acetylcholine Receptor by Cocaine and MK-801,” Proc. Nat. Acad. Sci. 97(25): 13895-13900 (2000), Chen et al., “Mechanism-Based Discovery of Small Molecules that Prevent Noncompetitive Inhibition by Cocaine and MK-801 Mediated by Two Different Sites on the Nicotinic Acetylcholine Receptor,” Biochemistry 43:10149-10156 (2004), Hess et al., “Reversing the Action of Noncompetitive Inhibitors (MK-801 and Cocaine) on a Protein (Nicotinic Acetylcholine Receptor)-Mediated Reaction,” Biochemistry 42:6106-6114 (2003), Cui et al., “Selection of 2′-Fluoro-modified RNA Aptamers for Alleviation of Cocaine and MK-801 Inhibition of the Nicotinic Acetylcholine Receptor,” J. Membrane Biol. 202:137-149 (2004), Sivaprakasam et al., “Minimal RNA Aptamer Sequences That Can Inhibit or Alleviate Noncompetitive Inhibition of the Muscle-Type Nicotinic Acetylcholine Receptor,” J. Membrane Biol. 233:1-12 (2010), Ulrich et al., “In Vitro Selection of RNA Molecules that Displace Cocaine from the Membrane-Bound Nicotinic Acetylcholine Receptor,” Proc. Nat. Acad. Sci. 95: 14051-14056 (1998), and Grewer et al., “On the Mechanism of Inhibition of the Nicotinic Acetylcholine Receptor by the Anticonvulsant MK-801 Investigated by Laser-Pulse Photolysis in the Microsecond-to-Millisecond Time Region,” Biochemistry 38(24):7837-46 (1999), which are all hereby incorporated by reference in their entirety).

Ligands that bind to the regulatory site of the nicotinic acetylcholine receptors comprise two different classes. Both classes modulate the opening and closing of the ion channel of the receptor to control flow of inorganic cations through the ion channel. Class 1 ligands are compounds that bind with higher affinity to the regulatory site on the closed-channel form than on the open-channel form of the receptor. Class 1 ligands facilitate closure and/or continued existing closure of the receptor ion channel, which inhibits neurotransmission. Class 1 ligands include both endogenous and exogenous compounds. Prototypical exogenous Class 1 ligands include, without limitation, cocaine, MK-801, and phencyclidine.

Broadly, Class 2 ligands are compounds that bind to the regulatory site on nicotinic acetylcholine receptors and shift the channel-opening equilibrium towards the open channel form of the receptor. For example, in the presence of an activating ligand such as acetylcholine or carbamoylcholine, in the presence of a deleterious factor such as a Class 1 ligand, a mutation, etc., Class 2 ligands bind with equal or higher affinity to the regulatory site on the open-channel form of the receptor than to the closed-channel form. This binding shifts the channel-opening equilibrium to the open-channel state and alleviates the inhibition and impairment caused by a Class 1 compound, mutation, etc.

Laboratory work has demonstrated beneficial effects of multiple Class 2 compounds on cell function in vitro. With the use of electrophysiological current-recording techniques, when specific neurotransmitter receptors in the plasma membrane of a cell are stimulated by carbamoylcholine (100 micromolar), a stable analog of the natural ligand acetylcholine, an increase and then a decay in the induced current are observed. The current reflects the movement of cations through the open receptor. The amplitude of this current is decreased in the presence of certain other compounds (designated as Class 1 compounds with prototypical examples cocaine and MK-801). The amplitude of the decreased current is increased when an alleviatory Class 2 compound is used to reverse the effect of the Class 1 compound (see Hess et al., “Reversing the Action of Noncompetitive Inhibitors (MK-801 and Cocaine) on a Protein (Nicotinic Acetylcholine Receptor)-Mediated Reaction,” Biochemistry 42:6106-6114 (2003), which is hereby incorporated by reference in its entirety).

Exemplary Class 2 ligands suitable for use in accordance with the methods of the present invention include, without limitation, tropane and its derivatives, e.g., ecgonine, ecgonine methyl ester, RTI-4229-70, RCS-III-143, RCS-III-140A, RCS-III-218, and RCS-III-202A, piperidine and its derivatives, derivatives of MK801 (but not MK-801), derivatives of phencyclidine (but not phencyclidine), and certain RNA aptamers all of which are described in more detail infra. These Class 2 ligands are the ligands that are suitable for use in the methods of the present invention to alleviate the toxic and addictive properties of abused and addictive drugs.

According to one embodiment of the present invention, a ligand that binds to nicotinic acetylcholine receptors and improves the condition of patients suffering from the effects drug addiction or poisoning comprises an organic compound that is a derivative or analogue of tropane. The general chemical structure of the tropane derivatives are as follows:

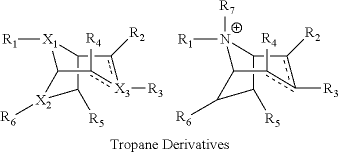

where R₁, R₂, R₃, R₄, R₅, R₆, and R₇ are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁, X₂, and X₃ are independently selected from the group consisting of N, S, O, and C.

In addition, other Class 2 ligands that bind to nicotinic acetylcholine receptors and improve addiction or poisoning states, include, but not limited to, the following organic compounds: ecgonine; ecgonine methyl ester; RTI-4229-70; RCS-III-143; RCS-III-140A; RCS-III-218; RCS-III-202A; and analogues and/or derivatives of these compounds.

As referred to herein, the organic compound “ecgonine” has the following chemical structure:

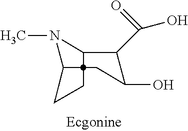

As referred to herein, the organic compound “ecgonine methyl ester” or “EME” has the following chemical structure:

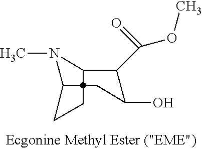

As referred to herein, the organic compound “RTI-4229-70” has the following chemical structure:

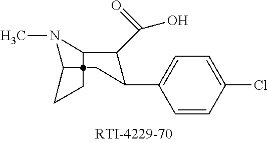 [SMILES: CN1C2CCC1C(C(=O)O)C(c1ccc(Cl)cc1)C2]

As referred to herein, the organic compound “RCS-III-143” has the following chemical structure:

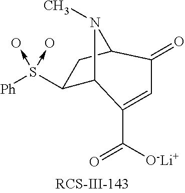

As referred to herein, the organic compound “RCS-III-140A” has the following chemical structure:

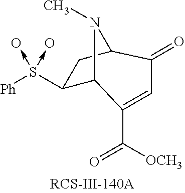 [SMILES: COC(=O)C1=CC(=O)C2CC(S(=O)(=O)c3ccccc3)C1N2C]

As referred to herein, the organic compound “RCS-III-218” has the following chemical structure:

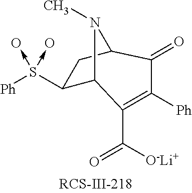

As referred to herein, the organic compound “RCS-III-202A” has the following chemical structure:

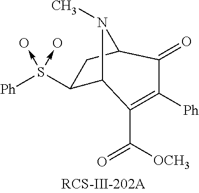 [SMILES: COC(=O)C1=C(c2ccccc2)C(=O)C2CC(S(=O)(=O)c3ccccc3)C1N2C]

In another embodiment of the present invention, ligands that bind to nicotinic acetylcholine receptors and are suitable for treatment and/or prevention of drug poisoning and addiction include one of more of the following cocaine analogs and derivatives:

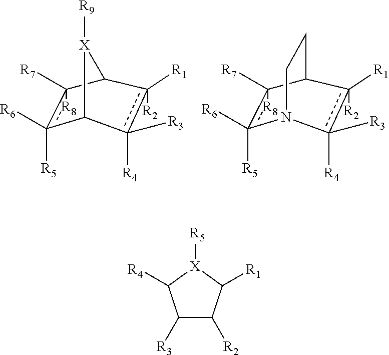

where R₁, R₂, R3, R₄, R₅, R₆, R₇ R₈ and R₉ are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁, X₂, and X₃ are independently selected from the group consisting of N, S, O, and C.

In another embodiment of the present invention, ligands that bind to nicotinic acetylcholine receptors and are suitable for treatment and/or prevention of drug poisoning and addiction include one of more of the following analogs and derivatives of piperidine as follows:

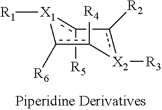

where R₁, R₂, R₃, R₄, R₅, and R₆, are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁, X₂, and X₃ are independently selected from the group consisting of N, S, O, and C.

In another embodiment of the present invention, ligands that bind to nicotinic acetylcholine receptors and are suitable for treatment and/or prevention of drug poisoning and addiction include one or more of the following analogs and derivatives of MK-801, with the proviso that the ligand is not dizocilpine. As referred to herein, the general chemical structures of these derivatives are as follows:

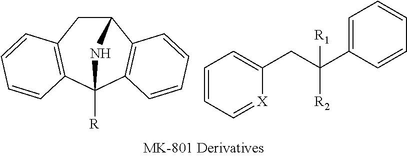

where R, R₁, and R₂, are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁, X₂, and X₃ are independently selected from the group consisting of N, S, O, and C.

In another embodiment of the present invention, ligands that bind to nicotinic acetylcholine receptors and are suitable for treatment and/or prevention of drug poisoning and addiction include one of more of the following analogs and derivatives of phencyclidine (PCP), with the proviso that the ligand is not PCP. As referred to herein, the general chemical structures of suitable PCP derivatives are as follows:

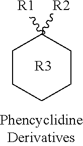

where

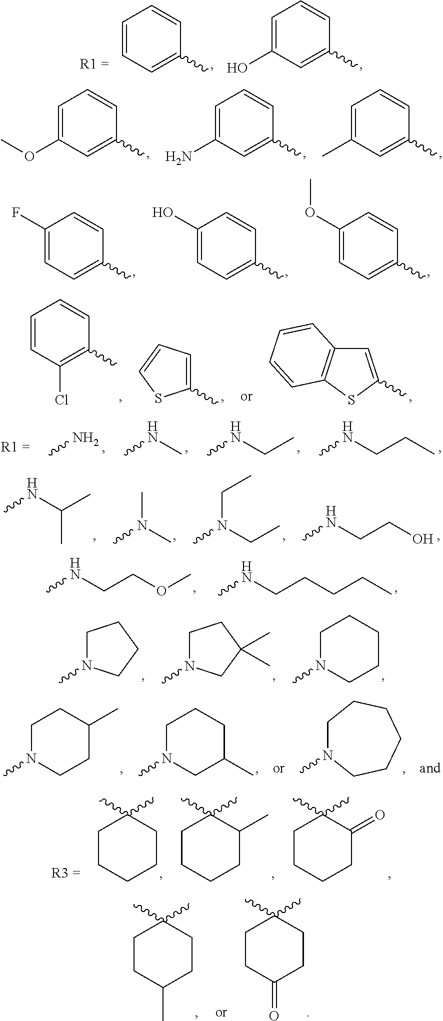

In another aspect, the present invention relates to a method of treating or preventing drug poisoning or drug addiction in a subject that involves administering to a subject having or at risk of having drug poisoning or drug addiction, an aptamer that binds to nicotinic acetylcholine receptors and improves, prevent, or treats the states of addiction or poisoning.

In one particular embodiment, Class 2 compounds reverse the poisonous effects of antimuscarinic anticholinergic drugs, such as atropine, scopolamine and hyoscine. EME, in one embodiment, reverses the effects of scopolamine. These and similar compounds, some of them constituents of belladonna, or “deadly nightshade” competitively antagonize the effects of acetylcholine at peripheral muscarinic receptors and, if they cross the blood-brain barrier, in the central nervous system. Although useful in medicine as pre-medicants, these compounds can cause death from excessive increase in heart rate, and/or central nervous system depression, and at lower doses cause distress from dry mouth and effects on the intestine and the eye. According to the discoveries of the present invention, Class 2 compounds act as novel “pro-cholinergics”, promoting return to normal of both peripheral and central cholinergic function inhibited by both antimuscarinic and antinicotinic compounds, such as atropine (peripheral antimuscarinic) and cocaine (central antinicotinic). The pharmacological properties of antimuscarinic and antinicotinic compounds are described in detail in such authoritative texts as Goodman and Gilman's, The Pharmacological Basis of Therapeutics 12^{th} edition (Lawrence L. Brunton, PhD, Bruce A. Chabner, M D, and Bjorn C. Knollmann, eds., McGraw-Hill 2011).

The hallmark of an anticholinergic compound (atropine, scopolamine, and also antihistamines and older antipsychotic drugs) is the moiety N—C—C—C—. In varying embodiments, the string of N—C—C—C— can be branched, substituted and/or truncated, but does not, usually, involve a double bond. In one embodiment, the ligands that bind to nicotinic acetylcholine receptors and are suitable for treatment and/or prevention of drug poisoning and addiction contain the following moiety:

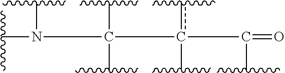

where [symbol]
can be a single or a double bond; and

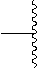

is the point of attachment of the moiety to the ligand.

In another embodiment, the ligands that bind to nicotinic acetylcholine receptors and are suitable for treatment and/or prevention of drug poisoning and addiction contain the following core structure:

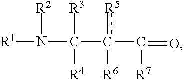

where [symbol]
can be a single or a double bond; R¹ can be H, C₁₋₆ alkyl, or aryl, wherein C₁₋₆ alkyl and aryl can be optionally substituted 1-3 times with —OH, halogen, or C₁₋₆ alkyl; R² can be H, C₁₋₆ alkyl, aryl,

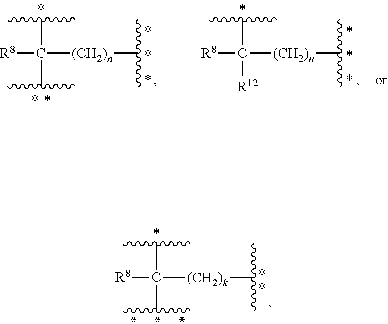

wherein C₁₋₆ alkyl and aryl can be optionally substituted 1-3 times with —OH, halogen, or C₁₋₆ alkyl; R³ can be H, —C₁₋₆ alkyl, —(CH₂)ₘ—, or —CR⁹R¹⁰—, wherein C₁₋₆ alkyl can be optionally substituted 1-3 times with —OH, halogen, or C₁₋₆ alkyl; R⁴ can be H, halogen, aryl, or —C₁₋₆ alkyl, wherein aryl can be optionally substituted 1-3 times with —OH, halogen, or C₁₋₆ alkyl; R⁵ can be H, C₁₋₆ alkyl, aryl, —CR⁹R¹⁰—, or ═C(R¹¹)—C(O)—, wherein C₁₋₆ alkyl and aryl can be optionally substituted 1-3 times with —OH, halogen, or C₁₋₆ alkyl; R⁶ can be H, halogen, aryl, or —C₁₋₆ alkyl, wherein aryl can be optionally substituted 1-3 times with —OH, halogen, or C₁₋₆ alkyl; R⁷ can be —OH, —OC₁₋₆ alkyl, —C₁₋₆ alkyl, aryl, —NR⁹R¹⁰, or —OM, wherein C₁₋₆ alkyl and aryl can be optionally substituted 1-3 times with —OH, halogen, or C₁₋₆ alkyl; R⁸ can be H, halogen, aryl, or —C₁₋₆ alkyl, wherein aryl can be optionally substituted 1-3 times with —OH, halogen, or C₁₋₆ alkyl; R⁹ can be H, halogen, aryl, or —C₁₋₆ alkyl, wherein aryl can be optionally substituted 1-3 times with —OH, halogen, or C₁₋₆ alkyl; R¹⁰ can be H, —OH, —C₁₋₆ alkyl, aryl or —SO₂Aryl, wherein C₁₋₆ alkyl, aryl, and —SO₂Aryl can be optionally substituted 1-3 times with —OH, halogen, or C₁₋₆ alkyl; R¹¹ can be H, aryl, wherein aryl can be optionally substituted 1-3 times with —OH, halogen, or C₁₋₆ alkyl; R¹² can be H, halogen, aryl, or —C₁₋₆ alkyl, wherein aryl can be optionally substituted 1-3 times with —OH, halogen, or C₁₋₆ alkyl; M is metal selected from the group consisting of Li, Na, or K; n is 0-3; m is 2-3; k is 0 or 1;

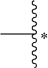

is the point of attachment to N;

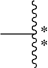

is the point of attachment to R²; and

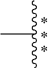

is the point of attachment to R⁵.

Scopolamine has this string on both sides of the tropane core structure. All of the disclosed Class 2 compounds have in their structures N—C—C—C═O, or something similar. Scopolamine (and also atropine) have a large substituent at the 3-position of the six-membered ring within the tropane core, apparently conferring sedative anticholinergic properties in the CNS. As will be readily apparent to those skilled in the art, polar compounds such as aptamers are likely to have only peripheral effects. Ecgonine, EME (cLogP=−1.83) and 3-acetoxy EME are the most polar of disclosed small molecule compounds, yet EME readily penetrates the brain. 3-Acetoxy EME was developed for its lesser polarity. Within the disclosed RCS series of compounds (i.e., RTI-4229-70; RCS-III-143; RCS-III-140A; RCS-III-218; RCS-III-202A; and analogues and/or derivatives of these compounds) (see Hess et al., “Reversing the Action of Noncompetitive Inhibitors (MK-801 and Cocaine) on a Protein (Nicotinic Acetylcholine Receptor)-Mediated Reaction,” Biochemistry 42:6106-6114 (2003), which is hereby incorporated by reference in its entirety), methyl esterification and phenyl substitution confer lesser polarity, leaving RCS-111-140A (cLogP=0.33; K_{D(alv)}0.8) as the least polar and the most potent, by some but not all standards. The RTI compound retains the core ecgonine structure but adds a strong electron withdrawing group, greatly reducing polarity (cLogP=2.66) and creating a highly potent (K_{D(alv)}=0.7) Class 2 compound. Thus, the present invention demonstrates how the molecules can be modified in chemical structure to suit the need for peripheral or central pharmacological action.

RNA aptamers are preferred types of nucleic acid elements that have specific affinity for a target molecule. Aptamers typically are generated and identified from a combinatorial library (typically in vitro) wherein a target molecule, generally, although not exclusively, a protein or nucleic acid is used to select from a combinatorial pool of molecules, generally although not exclusively oligonucleotides, those that are capable of binding to the target molecule. The term “aptamer” includes not only the primary aptamer in its original form, but also secondary aptamers derived from the primary aptamer (i.e., created by minimizing and/or modifying the structure of the primary aptamer). Aptamers, therefore, behave as ligands, binding to their target molecule.

Identifying primary aptamers basically involves selecting aptamers that bind a target molecule with sufficiently high affinity (e.g., K_{d}=20-50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell Biol.17 :1649-1657 (1997), which is hereby incorporated by reference in its entirety).

Any method known in the art can be used to identify primary aptamers of any particular target molecule. In one embodiment (but not the only method) of the present invention the established in vitro selection and amplification scheme, SELEX, can be used. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), which are hereby incorporated by reference in their entirety.

In the case of RNA aptamers, where the sequence of the RNA has been established, the RNA molecule can either be prepared synthetically or a DNA construct or an engineered gene capable of encoding such an RNA molecule can be prepared.

Suitable examples of RNA aptamers that can be used in the methods of the present invention, include, but are not limited to, RNA aptamers that have the consensus sequences:
(a) SEQ ID NO:1 (i.e., ACCG), SEQ ID NO:2 (i.e., UCCG), SEQ ID NO:3 (i.e., UUUACCG), SEQ ID NO:4 (i.e., UUCACCG), and/or SEQ ID NO:5 (i.e., UUCACCGUAAGG);
(b) SEQ ID NO:6 (i.e., AUCACCGUAAGG (see Aptamer B5)), SEQ ID NO:7 (i.e., UUUACCGUAAGG (see Aptamer B15)), SEQ ID NO:8 (i.e., UUUUCCGUAAGG (see Aptamer B19)), SEQ ID NO:9 (i.e., UUUACCGUAAGG (see Aptamer B27)), SEQ ID NO:10 (i.e., AUCACCGUAAGG (see Aptamer B28)), SEQ ID NO:11 (i.e., UCCACCGUAGAU (see Aptamer B36)), SEQ ID NO:12 (i.e., AUCACCGUAAGG (see Aptamer B44)), SEQ ID NO:13 (i.e., UUUACCGUAAGG (see Aptamer B55)), SEQ ID NO:14 (i.e., UCCACCGUAAGA (see Aptamer B59)), SEQ ID NO:15 (i.e., UCCACCGUAAGA (see Aptamer B61)), SEQ ID NO:16 (i.e., UUUACCGUAAGG (see Aptamer B64)), SEQ ID NO:17 (i.e., UUUACCGUAAGG (see Aptamer B65)), SEQ ID NO:18 (i.e., UUUACCGUAAGG (see Aptamer B69)), SEQ ID NO:19 (i.e., UCCACCGUAAGA (see Aptamer B76)), SEQ ID NO:20 (i.e., UUUUCCGUAAGG (see Aptamer B78)), SEQ ID NO:21 (i.e., UCCACCGUAAGA (see Aptamer B108)), SEQ ID NO:22 (i.e., UUUACCGUAAGG (see Aptamer B111)), and/or SEQ ID NO:23 (i.e., AUCACCGUAAGG (see Aptamer B124));
(c) SEQ ID NO:55 (i.e., GCUGAA);
(d) SEQ ID NO:66 (i.e., GAAAG); and/or
(e) SEQ ID NO:88 (i.e., GUUAAU).

Suitable examples of RNA aptamers that can be used in the methods of the present invention, include, but are not limited to, RNA aptamers having a nucleotide sequence selected from:
(a) SEQ ID NO:24 (Aptamer B5), SEQ ID NO:25 (Aptamer B15), SEQ ID NO:26 (Aptamer B19), SEQ ID NO:27 (Aptamer B27), SEQ ID NO:28 (Aptamer B28), SEQ ID NO:29 (Aptamer B36), SEQ ID NO:30 (Aptamer B44), SEQ ID NO:31 (Aptamer B55), SEQ ID NO:32 (Aptamer B59), SEQ ID NO:33 (Aptamer B61), SEQ ID NO:34 (Aptamer B64), SEQ ID NO:35 (Aptamer B65), SEQ ID NO:36 (Aptamer B69), SEQ ID NO:37 (Aptamer B76), SEQ ID NO:38 (Aptamer B78), SEQ ID NO:39 (Aptamer B108), SEQ ID NO:40 (Aptamer B111), and/or SEQ ID NO:41 (Aptamer B124);
(b) SEQ ID NO:42 (Aptamer 01), SEQ ID NO:43 (Aptamer 05), SEQ ID NO:44 (Aptamer 06), SEQ ID NO:45 (Aptamer 07), SEQ ID NO:46 (Aptamer 09), SEQ ID NO:47 (Aptamer 11), SEQ ID NO:48 (Aptamer 13), SEQ ID NO:49 (Aptamer 14), SEQ ID NO:50 (Aptamer 16), SEQ ID NO:51 (Aptamer 18), SEQ ID NO:52 (Aptamer 19), SEQ ID NO:53 (Aptamer 20,21), and/or SEQ ID NO:54 (Aptamer 22);
(c) SEQ ID NO:56 (Aptamer 3), SEQ ID NO:57 (Aptamer 8), SEQ ID NO:58 (Aptamer 23), SEQ ID NO:59 (Aptamer 24), SEQ ID NO:60 (Aptamer 26), SEQ ID NO:61 (Aptamer 30), SEQ ID NO:62 (Aptamer 31), SEQ ID NO:63 (Aptamer 38), SEQ ID NO:64 (Aptamer 39), and/or SEQ ID NO:65 (Aptamer 42);
(d) SEQ ID NO:67 (Aptamer 51), SEQ ID NO:68 (Aptamer S 13), SEQ ID NO:69 (Aptamer S14), SEQ ID NO:70 (Aptamer S21), SEQ ID NO:71 (Aptamer S24), SEQ ID NO:72 (Aptamer S29), SEQ ID NO:73 (Aptamer S43), SEQ ID NO:74 (Aptamer S44), SEQ ID NO:75 (Aptamer S45), SEQ ID NO:76 (Aptamer S46), SEQ ID NO:77 (Aptamer S47), SEQ ID NO:78 (Aptamer S49), SEQ ID NO:79 (Aptamer S50), SEQ ID NO:80 (Aptamer S53), SEQ ID NO:81 (Aptamer S56), SEQ ID NO:82 (Aptamer S59), SEQ ID NO:83 (Aptamer S62), SEQ ID NO:84 (Aptamer S15), SEQ ID NO:85 (Aptamer S17), SEQ ID NO:86 (Aptamer S28), and/or SEQ ID NO:87 (Aptamer S54); and/or
(e) SEQ ID NO:89 (Aptamer S5), SEQ ID NO:90 (Aptamer S18), SEQ ID NO:91 (Aptamer S20), SEQ ID NO:92 (Aptamer S25), SEQ ID NO:93 (Aptamer S48), SEQ ID NO:94 (Aptamer S51), and/or SEQ ID NO:95 (Aptamer S57).

Also included within the invention are modified aptamers, having improved properties such as decreased size, enhanced stability, or enhanced binding affinity. Such modifications of aptamer sequences include adding, deleting or substituting nucleotide residues, and/or chemically modifying one or more residues. Methods for producing such modified aptamers are known in the art and described in, e.g., U.S. Pat. No. 5,817,785 to Gold et al., and U.S. Pat. No. 5,958,691 to Wolfgang et al., which are hereby incorporated by reference in their entirety.

Chemically modified aptamers include those containing one or more modified bases. For example, modified pyrimidine bases may have substitutions of the general formula 5′-X and/or 2′-Y, and modified purine bases may have modifications of the general formula 8′-X and/or 2′-Y. The group X includes the halogens I, Br, Cl, or an azide or amino group. The group Y includes an amino group, fluorine, or a methoxy group. Other functional substitutions that would serve the same function may also be included. The aptamers of the present invention may have one or more X-modified bases, or one or more Y-modified bases, or a combination of X- and Y-modified bases. The present invention encompasses derivatives of these substituted pyrimidines and purines such as 5′-triphosphates, and 5′-dimethoxytrityl, 3′-beta-cyanoethyl, N,N-diisopropyl phosphoramidites with isobutyryl protected bases in the case of adenosine and guanosine, or acyl protection in the case of cytosine. Further included in the present invention are aptamers bearing nucleotide analogs, e.g., nucleotide analogs modified at the 5 and 2′ positions, including 5-(3-aminoallyl)uridine triphosphate (5-AA-UTP), 5-(3-aminoallyl) deoxyuridine triphosphate (5-AA-dUTP), 5-fluorescein-12-uridine triphosphate (5-F-12-UTP), 5-digoxygenin-11-uridine triphosphate (5-Dig-11-UTP), 5-bromouridine triphosphate (5-Br-UTP), 2′-amino-uridine triphosphate (2′-NH₂-UTP) and 2′-amino-cytidine triphosphate (2′-NH₂-CTP), 2′-fluoro-cytidine triphosphate (2′-F-CTP), and 2′-fluoro-uridine triphosphate (2′-F-UTP).

The aptamers may also be modified by capping at the 3′ and 5′ end and by inclusion of a modified nucleotide. For example, the aptamer can be modified by adding to an end a polyethyleneglycol, amino acid, peptide, inverted dT, nucleic acid, nucleosides, myristoyl, lithocolic-oleyl, docosanyl, lauroyl, stearoyl, palmitoyl, oleoyl, linoleoyl, other lipids, steroids, cholesterol, caffeine, vitamins, pigments, fluorescent substances, toxin, enzymes, radioactive substance, biotin and the like. For such alterations, see for example, U.S. Patent Publication No. 2005/0096290 to Adamis et al. and U.S. Pat. No. 5,660,985 to Wolfgang et al., which are hereby incorporated by reference in their entirety.

The sequences (consensus and RNA aptamer nucleotide sequences) referenced above by “SEQ ID NO.” are identified herein below in Tables 1, 2, 3, 4, 5, and 6.

TABLE 1
Consensus Regions of Selected RNA Aptamers

| RELATED | | |
|---|---|---|
| APTAMER | CONSENSUS REGION | SEQ ID NO: |
| Consensus | ACCG | 1 |
| Consensus | UCCG | 2 |
| Consensus | UUUACCG | 3 |
| Consensus | UUCACCG | 4 |
| Consensus | UUCACCGUAAGG | 5 |
| B5 | AUCACCGUAAGG | 6 |
| B15 | UUUACCGUAAGG | 7 |
| B19 | UUUUCCGUAAGG | 8 |
| B27 | UUUACCGUAAGG | 9 |
| B28 | AUCACCGUAAGG | 10 |
| B36 | UCCACCGUAGAU | 11 |
| B44 | AUCACCGUAAGG | 12 |
| B55 | UUUACCGUAAGG | 13 |
| B59 | UCCACCGUAAGA | 14 |
| B61 | UCCACCGUAAGA (B61) | 15 |
| B64 | UUUACCGUAAGG (B64) | 16 |
| B65 | UUUACCGUAAGG (B65) | 17 |
| B69 | UUUACCGUAAGG (B69) | 18 |
| B76 | UCCACCGUAAGA (B76) | 19 |
| B78 | UUUUCCGUAAGG (B78) | 20 |
| B108 | UCCACCGUAAGA (B108) | 21 |
| B111 | UUUACCGUAAGG (B111) | 22 |
| B124 | AUCACCGUAAGG (B124) | 23 |

TABLE 2
Selected RNA Aptamers

| | | SEQ |
|---|---|---|
| AP- | | ID |
| TAMER | SEQUENCE | NO: |
| B5 | 5′-CUCGAUCACCGUAAGGACAUCUACGUAAGUGUAAU | 24 |
| | GCGGCUUGUUUUCCCCAUGCGUCUGCAUAUCUGUU-3′ | |
| B15 | 5′-UUUACCGUAAGGCCUGUCAUCGUUUGACAGCGGCU | 25 |
| | UGUUGACCCUUCCACUAUGUGUGCCUGUAAUG-3′ | |
| B19 | 5′-ACUUCGUCUUGCAGCGCGGCUUGUCUCUUCCCACAU | 26 |
| | CCGUUCUAUCGGUAUGACUCUUUUUCCGUAAGGUCA-3′ | |
| B27 | 5′-UUUACCGUAAGGCCUGUCUUCGUUUGACAGCGGCU | 27 |
| | UGUUGACCCUCACACUUUGUACCUCUGCCUG-3′ | |
| B28 | 5′-CUCGAUCACCGUAAGGACAUCUACAUAAGUGUAAU | 28 |
| | GCGGCUUGUUUUCCCCAUGCAUCUGCAUAUCUGU-3′ | |
| B36 | 5′-UGUCCACCGUAGAUUGUAAACUAUCGCGUAAAGCG | 29 |
| | AAGUUUAUGUGGCUUGUUUUCCCACGCCUUG-3′ | |
| B44 | 5′-CUCGAUCACCGUAAGGACAUUUACGUAAGUGUAAU | 30 |
| | GCGGCUUGUUUUCCCCAUGCGUCUGCAUAUCUGU-3′ | |
| B55 | 5′-UUUACCGUAAGGCCUGUCUUCGUUUGACAGCGGCU | 31 |
| | UGUUGACCCUCACACUUUGUACCUGCUGCCAA-3′ | |
| B59 | 5′-UCCACCGUAAGAUUGUAAACUAUCGGGUAAAGACG | 32 |
| | AAGUUUAUGUGGCUUGUUUCCCACCGCCUUGCC-3′ | |
| B61 | 5′-UGUCCACCGUAAGAUUGUAAACUAUCGUAAAGACG | 33 |
| | AAGUUUAUGUGGCUUGUUUUCCCACCGCCUUGCC-3′ | |
| B64 | 5′-UUUACCGUAAGGCCUGUCAUCGUUUGACAGCGGCU | 34 |
| | UGUUGACCCUUCCACUAUGUGUGCCUGUAAUG-3′ | |
| B65 | 5′-UUUACCGUAAGGCCUGUCUUCGUUUGACAGCGGCU | 35 |
| | UGUUGACCCACACACUUUGUCCCGGCUGCAG-3′ | |
| B69 | 5′-UUUACCGUAAGGCCUGUCUUCUUUUGACAGCGGCU | 36 |
| | UGUUGACCCUCACGCUUUGUCCCUGCUGUACCUG-3′ | |
| B76 | 5′-UCCACCGUAAGAUUGUAAACUAUCGCGUAAAAGAC | 37 |
| | GAAGUUUAUGUGGCUUGUUUUCCCACCGCCUUG-3′ | |
| B78 | 5′-ACUUCGUCUUGCAGCGCGGCUUGUCUUCCCACAUC | 38 |
| | CGUUCUAUCGGUAUGACUUUUUCCGUAAGGUCA-3′ | |
| B108 | 5′-UCCACCGUAAGAUUGUAAACUAUCGCGUAAAGACG | 39 |
| | AAGUUUAUGUGGCUUGUUUCCCACCACCUUGCG-3′ | |
| B111 | 5′-UUUACCGUAAGGCCUGUCUUCGUUUGACAGCGGCU | 40 |
| | UGUUGACCCUCACGCUUUGUCCCAUGCCCGUC-3′ | |
| B124 | 5′-CUCGAUCACCGUAAGGACAUUUACGUAAGUGUAAU | 41 |
| | GCGGCUUGUUUUCCCCAUGCGUUUGCAUAUCUGUG-3′ | |

TABLE 3
Sequences of Selected RNA Aptamers that Bind to
the Nicotinic Acetylcholine Receptors

| | | SEQ ID |
|---|---|---|
| APTAMER | SEQUENCE | NO: |
| 01 | ACGUUGAGUACAACCCCACCCCGUUCACGGUAGC | 42 |
| | CCUGUA | |
| 05 | GCUACAGUACAACGGGCCGUGUGGAAUACACCGA | 43 |
| | CAAGG | |
| 06 | UCCACCGAUCUAGAUGAUCCAGGCACCCGACCAC | 44 |
| | CACCUC | |
| 07 | GCUUGUGGACCAAGAAGCAACCAGUCACCGUUGC | 45 |
| | CCC | |
| 09 | CAACAGUCCUGUGUCCGUUGAAUCCUCUAGAUCC | 46 |
| | AGGGUG | |
| 11 | GGACCCCCCACAGCAAGUUUGCCGGCGACCGCGU | 47 |
| | UCUUG | |
| 13 | CUUGCCACUCCUGUCUAGCUGGCGUAGACCGCGC | 48 |
| | AGAAAG | |
| 14 | GCUAGUAGCCUCAGCAGCAUAGUUUCGCCGCUAU | 49 |
| | GCAGUA | |
| 16 | UAGCAUAAUGUGGAGCGUUGACCGGACCUCUCCA | 50 |
| | GUCGUA | |
| 18 | UGGACUACGCACCCGCUAGUCCGUCCAAGAACUG | 51 |
| | UGCG | |
| 19 | UUCUGUUCCGACCAAUUGAAUAGUCACCGUGAUG | 52 |
| | AUUUGA | |
| 20, 21 | GAUGCCAGCGCGCAUUCUUCACCGAAGUACGUAU | 53 |
| | CCACG | |
| 22 | UUCGCCGCUGCACUCUCGCAGCACUGGUCGGGAU | 54 |
| | GUGUC | |

TABLE 4
Sequences of RNA Aptamers that Alleviate
Inhibition of Nicotinic Acetylcholine Receptors

| | | SEQ |
|---|---|---|
| | | ID |
| APTAMER | SEQUENCE | NO: |
| Consensus | GCUGAA | 55 |
| 3 | AGUAGGAAUACCCCCAUCCAAAGCUCGCUA | 56 |
| | GGCUGAACAC | |
| 8 | GACGGCCCGAGAUUGCAGAAAAACGCGCCC | 57 |
| | ACGUGUCAGA | |
| 23 | UCCCUAGCUGACGAUGGAUCUUGGAUCACA | 58 |
| | UAGGCUGCGC | |
| 24 | GGACAUGCCGGUCUUGAGCGGAGGUGAACC | 59 |
| | GUACCACG | |
| 26 | AACACGCCUCAGGACGCCAGGUGAACCCUC | 60 |
| | GAACC | |
| 30 | AACGCUGAAUCCCCCGGUCAUAGAACUUUG | 61 |
| | AUAGUACAG | |
| 31 | UACUGAAUGAUCUCCACCCGCCGGAAUGCG | 62 |
| | UAUAGUCCCU | |
| 38 | GCUGGGGAAAGCAGGUCCGUUCCCACCGCC | 63 |
| | UGAAGCUUUG | |
| 39 | CCUCCUGACACAACCACCCAACCACCUUCU | 64 |
| | UGAAACAUUU | |
| 42 | ACAACCUUGAUUGCUUGAAACCUCUAACCC | 65 |
| | GAGGCUCUGUA | |

TABLE 5
Sequences of 2′-Fluoropyrimidine-Modified RNA
Aptamers that Bind to Nicotinic
Acetylcholine Receptors

| | | SEQ |
|---|---|---|
| APTAMER | SEQUENCE | ID NO: |
| Consensus | GAAAG | 66 |
| S1 | CGAACGUGGACGAAGGGCGGUUUGUGAGUGC | 67 |
| | UUA | |
| S13 | CUGACUGCGUCUCUAUAUGACAUAGGCGAUG | 68 |
| | AGAAAGCAGA | |
| S14 | GGAACAGACGUCAUCUGUGGCACGUCCGCUG | 69 |
| | CUAGCAGAGA | |
| S21 | GACACAAGCUGGACCACGUCAAGCGUUUUGU | 70 |
| | GAAAGCAGGU | |
| S24 | UGGCAUCUUGUGCAUGACAACAGAGGGUGAA | 71 |
| | ACCAACGGGU | |
| S29 | AAACUUGCCUUGGUUUAUAACGUAACAAUAC | 72 |
| | AGAACGA | |
| S43 | AGAAUCUAAGACGUGAAAAUGGUAAGACAUU | 73 |
| | CUCUACC | |
| S44 | AGGUGUGCGCAGACGAAUAGGGUUGUGCGAA | 74 |
| | AGUCUAGCA | |
| S45 | UUUAGAGUUGAAAUGCGUAAUGGUUAAAUGA | 75 |
| | UCCAUUCUG | |
| S46 | AACAAUGCGAGGGUAAAAGCAUGUUCUAACC | 76 |
| | AGGGAGGGA | |
| S47 | AUGGAAGCCCUUGAUUCUACGGAUCUAGCGA | 77 |
| | GAUUU | |
| S49 | CCUGUAAGGGCGAAACUAAGCGAGAAAUCAU | 78 |
| | UAGGAUGA | |
| S50 | CUCAAUGCAUACGCUGGUCAACGGGACGAUU | 79 |
| | AGUGACAAGGCCGC | |
| S53 | AAUAAGUGGCAAGUAGCCUAGAGAUUAGAAG | 80 |
| | ACCUCAAC | |
| S56 | AAUUGACGAGCUGGUGGGAGAUAGUCUCAGG | 81 |
| | UAUCUUGUGC | |
| S59 | GGUGGACAGUAACUCCUUAGAUGCGGUAGAU | 82 |
| | UCGUAGC | |
| S62 | UACGCGCUUAUGAUAAAGGGUUAGAAGGACG | 83 |
| | AGCGUCGCA | |
| S15 | CACAUGCAGAGUAGUGUAAGGUAACACCCAG | 84 |
| | GUUUUUUG | |
| S17 | CCGGGGCGCAGGUGUCCCUGACGAUGAUCAA | 85 |
| | UUUCGGGUGA | |
| S28 | GACGCCUUUAUGAAUGACCAGGGAAGUUGUC | 86 |
| | AGAAGAGG | |
| S54 | GUCACUUUCUGAAUGGGAGAUAUCUUCGAUA | 87 |
| | UGGUAAU | |

TABLE 6
Sequences of 2′-Fluoropyrimidine-Modified RNA
Aptamers that Alleviate Inhibition of the
Nicotinic Acetylcholine Receptors

| | | SEQ |
|---|---|---|
| APTAMER | SEQUENCE | ID NO: |
| Consensus | GUUAAU | 88 |
| S5 | GAAGGCGAAAGGCACAAAGAUCUGAUGAAGU | 89 |
| | UAAUGGAUCA | |
| S18 | GUUAAUCGCUGAAUAUUCGAAGUGCUUUCCG | 90 |
| | UGAU | |
| S20 | UGGGCUUAGGUGUUAAGUCGAUGACUGUUCA | 91 |
| | UUCUCGGUA | |
| S25 | ACGUGAGCGAGCAAUAAAAGUCCCCUGGGGC | 92 |
| | GGAGUUAAA | |
| S48 | GGGAGAGUCUACGGAUCCUAGAAAAAGCAGG | 93 |
| | ACGUUAUU | |
| S51 | CAAAGGGGAGCCACGGGGCGACGUGUAAUCC | 94 |
| | UCUAUUCAGCA | |
| S57 | AAUGAAGGCAAUUCUUUAACGUUAAUAGGAA | 95 |
| | GGGGGUAAA | |

In one particularly preferred embodiment of the present invention, the pro-cholinergic compounds are natural or semisynthetic aptamers, that may or may not be truncated, containing one or more uridine residues which may or may not be substituted at various atomic locations in accord with the chemotype. In this embodiment, the minimal sequence for Class 2 activity has been shown to be GCUG, illustrating the significance of uridine (U), which incorporates the string

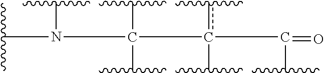

where [symbol]
can be a single or a double bond; and

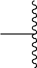

is the point of attachment of the moiety to the ligand, now known to be the key chemotype for Class 2 activity in both aptamers and small molecules.

The nicotinic acetylcholine receptor ligands that are suitable for the treatment and/or prevention of drug poisoning or drug addiction of the present invention can be administered orally, parenterally, for example, subcutaneously, intravenously, intramuscularly, intracerebroventricularly, intraparenchymal (i.e., brain or brain stem), intravascularly, intraperitoneally, by intranasal inhalation, or by application to mucous membranes, such as, that of the nose, throat, and bronchial tubes. The ligands may be administered alone or with suitable pharmaceutical carriers, and can be in solid or liquid form such as, tablets, capsules, powders, solutions, suspensions, or emulsions.

The nicotinic acetylcholine receptor ligands that treat or prevent drug poisoning or drug addiction of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, the small molecule and aptamer ligands of the present invention may be incorporated with excipients and used in the form of tablets, capsules, elixirs, suspensions, syrups, and the like. Such compositions and preparations should contain at least 0.1% of active compound. The percentage of nicotinic acetylcholine receptor ligand in these compositions may, of course, be varied and may conveniently be between about 2% to about 60% of the weight of the unit. The concentration of nicotinic acetylcholine receptor ligand in such therapeutically useful composition is such that a suitable dosage will be obtained. Preferred compositions according to the present invention are prepared so that an oral dosage unit contains between about 1 and 250 mg of one or more nicotinic acetylcholine receptor ligands of the present invention.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The nicotinic acetylcholine receptor ligands of the present invention may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

Pharmaceutical forms of the nicotinic acetylcholine receptor ligands of the present invention that are suitable for injectable use include sterile aqueous solutions or dispersions and sterile powders for the extemporaneous preparation of sterile injectable solutions or dispersions. In all cases, the form must be sterile and must be fluid to the extent that easy use in syringes exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The nicotinic acetylcholine receptor ligands of the present invention may also be administered directly to the airways in the form of an aerosol. For use as aerosols, the ligands of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The following examples illustrate the broadly-accepted logic and process of drug product discovery and development, a process which utilizes five broad scientific extrapolations: (i) from organic chemical structure to pharmacological receptor interaction; (ii) from in vitro to in vivo observations; (iii) from physico-chemical properties to pharmacokinetic properties; (iv) from animals to humans in vivo; and (v) from healthy human volunteers to sick patients. This process and its reliability are exemplified in multiple reference texts, notably Goodman and Gilman's, The Pharmacological Basis of Therapeutics 12^{th} edition (Lawrence L. Brunton, PhD, Bruce A. Chabner, M D, and Björn C. Knollmann, eds., McGraw-Hill 2011), Drug Disposition and Therapeutics (Curry et al.), and Edwards Principles and Practice of Pharmaceutical Medicine (3^{rd} ed'n) (Lionel D. Edwards, Andrew J. Fletcher, Anthony W. Fox, and Peter D. Stonier, eds., Wiley-Blackwell 2011), and references cited therein, which are hereby incorporated by reference in their entirety.

### EXAMPLES

The following examples are provided to illustrate embodiments of the present invention but are by no means intended to limit its scope.

### Example 1

### Morris Water Maze Probe Trial With Scopolamine

The Morris water maze for rats (San Diego Instruments) uses a 70 inch diameter swimming tank, in which rats, one at a time, are placed to determine the swimming time taken to find a platform, which can be visible or submerged, and is placed randomly in the tank, with its position locatable by means of navigation in response to visible clues. A video camera is positioned to record the swimming path of the rat, and computer analysis of the path permits accurate assessment of elapsed time, distance traveled, and route taken to achieve particular objectives. This technique is used to study memory, learning and spatial working, in healthy, diseased, and drug-affected states. The test can be applied acutely (probe test) or can involve considerable training and repeat measure experimental designs. The term “latency” in the context of the Morris water maze is used to depict the relation of time taken to escape from the water (Morris R., “Development of a Water-Maze Procedure for Studying Spatial Learning in The Rat,” Journal of Neuroscience Methods 11:47-60 (1984) and Wongwitdecha et al., “Effects of Social Isolation Rearing on Learning in the Morris Water Maze,” Brain Research 715:119-124 (1996), both of which are hereby incorporated by reference in their entirety).

This was a single dose probe trial in normal healthy rats with the objective of determining whether: (a) there is an effect in raising cognitive efficiency in unimpaired rats and (b) impairment caused by scopolamine can be reversed by EME.

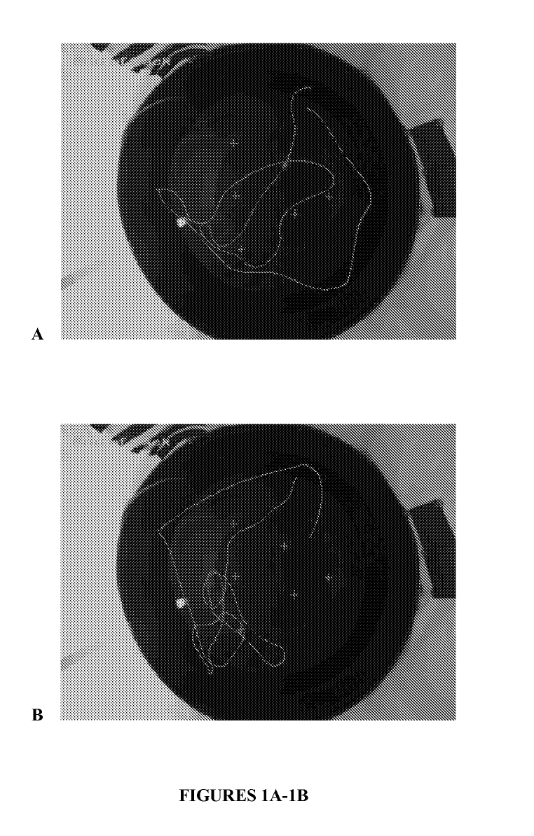

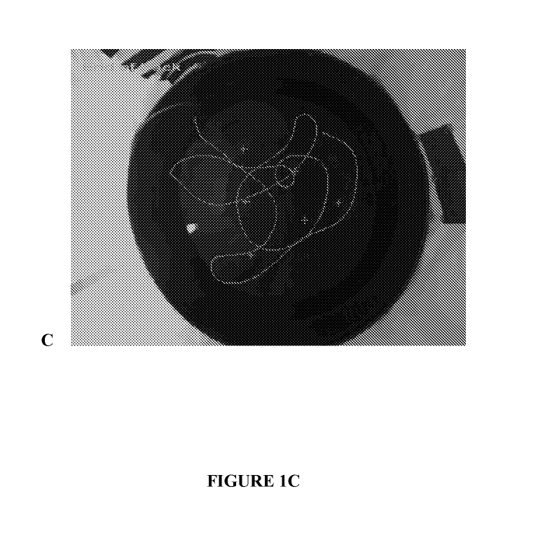

FIGS. 1A-1C show three individual swimming traces for rats treated with a control vehicle (FIG. 1A), a combined dose of ecgonine methyl ester (EME) (10 mg/kg) and scopolamine (1 mg/kg) (FIG. 1B), and a single dose of scopolamine (1 mg/kg) (FIG. 1C). The target area was in the “south west” or bottom left quadrant of the bath. The control rat found the target area. The scopolamine treated rat showed no preference, and the rat treated with the combination found the target area. In the experiment as a whole, the group size was 10, and analysis of variance showed that there was no difference between the EME group and the controls, but also no difference between the EME/scopolamine combination group and controls or the EME group. The scopolamine only group, however, showed the impairment previously shown by many groups of investigators over many years. This observation was significant (p<0.05). It can be concluded that EME reversed the scopolamine effect but had no effect on its own.

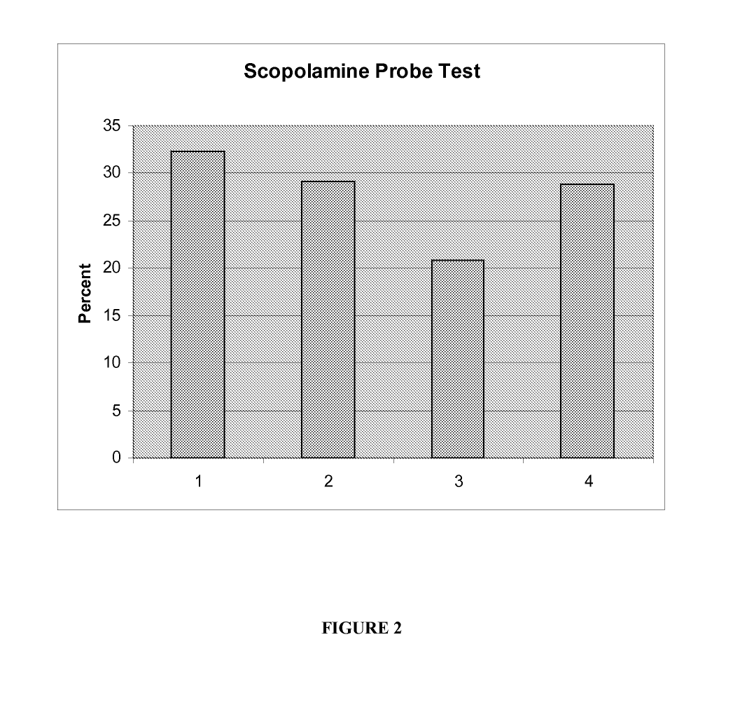

The group results in this probe trial with vehicle (VEH), EME alone, scopolamine alone, and EME plus scopolamine in the acute dose Morris water maze experiment are shown in FIG. 2. In particular, column 1 depicts the probe test results for the vehicle, column 2 depicts probe test results for EME alone, column 3 depicts probe test results for scopolamine alone, and column 4 depicts probe test results for EME in combination with scopolamine. Notably, EME was found to restore function impaired by scopolamine.

### Example 2

### Molecular Modeling Relevant to Blood-Brain Barrier Transfer

Molecular modeling calculations (cLog P—Hansch and Ghose/Crippen, and H-bond donor and acceptor, plus hydrogen bonding—Hansen) were conducted along with an analysis of related data to relevant kinetic constants using the compounds of the present application, and with scopolamine and other comparators. These molecular modeling calculations were compared to chemical structures of the compounds, which are shown in Table 7, infra.

TABLE 7
Properties of a Collection of Disclosed Compounds

| | | Log P | H-Bond | | |
|---|---|---|---|---|---|
| | | (Ghose | Donors | Hydrogen | |
| | Log P | and | and | Bonding | K_{D(Alv)} |
| Compound | (Hansch) | Crippen) | Acceptors | (Hansen) | (μM) |
| Cocaine | 1.642 | 1.925 | 0 | 6.7 | N/A |
| Ecgonine | −1.453 | −0.117 | 1 | 13.3 | 0.8-12.7 |
| methyl | | | | | |
| ester (EME) | | | | | |
| Ecgonine | −1.829 | −0.148 | 2 | 15.7 | 3.5 |
| RTI Compound | 2.659 | 2.886 | 1 | 8.7 | 0.7-14 |
| 3.Acetoxy | −3.774 | 1.443 | 0 | 10.5 | 3.3 |
| EME | | | | | |
| RCS-111-218 | −4.36 | 1.421 | 1 | 13.0 | 5 |
| RCS-111-202A | −2.38 | 2.948 | 0 | 9.5 | 2.8 |
| RCS-111-143 | −2.96 | 2.916 | 1 | 11.6 | 3.3 |
| RCS-111-140A | −.0.327 | 0.0124 | 0 | 8.8 | 0.8-8.2 |

The disclosed physicochemical data in Table 7 are derived from Molecular Modeling Pro. The values for the alleviatory dissociation constant (K_{D(Alv)}) shown for alleviation of Class 1 compound effects by Class 2 compounds are dependent on the identity and concentration of the Class 1 compound used, and where effects of multiple Class 1 compounds have been alleviated, a range is given. They arise from equations that link A₀ and A_{I} (the corrected currents measured in the absence and presence of the inhibitor, respectively), I₀ (the inhibitor concentration), K₁(obs) (the dissociation constant of the inhibitor from the rapidly equilibrating inhibitory site), K_{D(Alv)} (the dissociation constant of the compound that alleviates inhibition of the rapidly equilibrating site), and the concentration of the alleviatory compound. Such equations are used to fit the curve and calculate K_{I}(obs) and K_{D(Alv)} (Hess et al., “Mechanism-Based Discovery of Ligands that Counteract Inhibition of the Nicotinic Acetylcholine Receptor by Cocaine and MK-801,” Proc. Nat. Acad. Sci. 97(25): 13895-13900 (2000), which is hereby incorporated by reference in its entirety). The equation assumes a competitive mechanism between the inhibitor and a compound that alleviates inhibition. However, this does not refer to a competition between the activating ligand (acetylcholine or carbamoylcholine) and the inhibitor or modulatory ligand—for further details see Chen et al., “Mechanism-Based Discovery of Small Molecules that Prevent Noncompetitive Inhibition by Cocaine and MK-801 Mediated by Two Different Sites on the Nicotinic Acetylcholine Receptor,” Biochemistry 43:10149-10156 (2004), which is hereby incorporated by reference in its entirety.

### Example 3

### Brain and Plasma Concentrations of Ecgonine Methyl Ester 10 mg/kg Intraperitoneal Doses in Rats

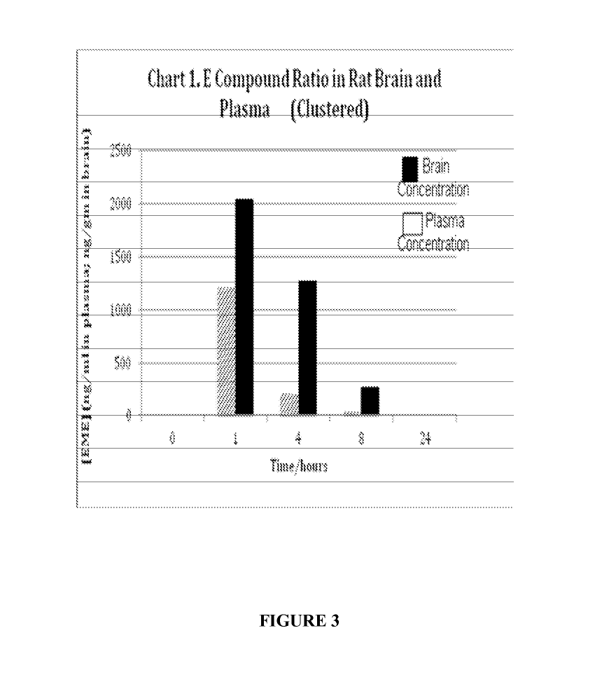

Plasma and Brain Concentrations after Intraperitoneal Doses—Twenty young adult rats in groups of four were given intraperitoneal doses of 10 mg/kg, and killed and dissected at various times after dosing. Brain and plasma concentrations were assessed by GC-MS. Samples were pre-dose, and at 1, 2, 4 and 24 hours after the dose. The data are shown in the FIG. 3. Each point is the mean value from four rats. The tested compound is referred to in FIG. 3 as both “EME” and “E Compound”.

Plasma concentrations had already peaked at one hour—brain concentrations were maximal at 2 hours. The maximum brain-to-plasma ratio was approximately 10. The data show rapid absorption after the IP dose, a biexponential decay of plasma concentrations, as if the drug confers on the body the characteristics of a two-compartment system, and sufficient persistence in the body to predict a half-life in humans of 6-8 hours.

### Example 4

### Ecgonine Methyl Ester (EME) Protects Against Cocaine Lethality in Mice

Table 8, infra (adapted from Hoffman et al., “Ecgonine Methyl Ester Protects Against Cocaine Lethality in Mice,” J. Toxicol. Clin Toxicol. 42(4):349-54 (2004), which is hereby incorporated by reference in its entirety) shows the results of an in vivo test of the ability of a Class 2 small molecule to reverse the toxicity of cocaine, a Class 1 small molecule, with both compounds crossing the blood-brain barrier. In particular, using a randomized blinded protocol, 80 mice were pretreated with either ecgonine methyl ester (EME) (50 mg/kg) or 0.9% sodium chloride solution. Five minutes later, all animals received 126 mg/kg of cocaine and were observed for seizures and death. Pretreatment with ecgonine methyl ester (EME) increased survival, but had no significant effect on times to seizure and death in those animals not protected.

TABLE 8
EME Protection Against Cocaine Lethality in Mice

| Measurement | Control | EME Pre-Treated | Statistics |
|---|---|---|---|
| Survival | 2/40 | 9/40 | P < 0.05 |
| Time to Seizure | 1.5 min | 2.0 min | P > 0.05 |
| Time to Death | 4.6 min | 4.5 min | P > 0.05 |
| N for seizure and death | 38 | 31 | |

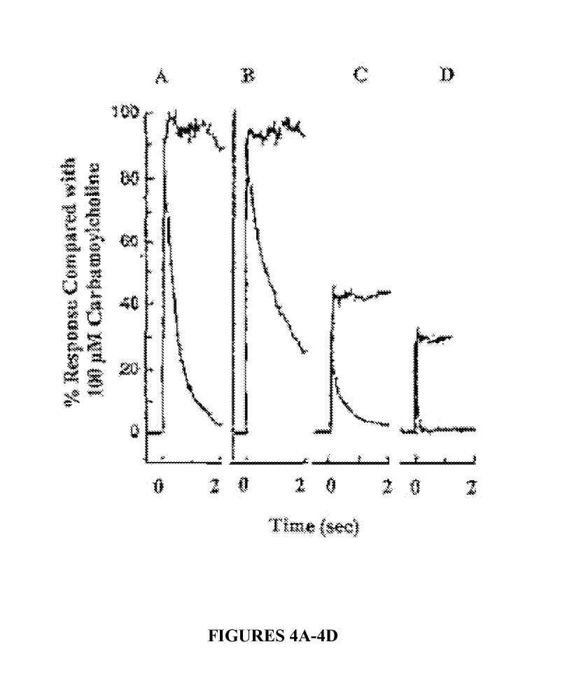

Several prior studies have tested the in vitro effects of Class 1 and Class 2 aptamers on nicotinic acetylcholine receptor ligands. For example, the in vitro effects on BC₃H1 nicotinic acetylcholine receptor currents of a single-cloned Class 1 or Class 2 RNA aptamer or cocaine in the presence of carbamoylcholine are shown in in FIGS. 4-6 (in part adapted Ulrich et al., “In Vitro Selection of RNA Molecules that Displace Cocaine From the Membrane-Bound Nicotinic Acetylcholine Receptor,” Proc. Nat. Acad. Sci. 95: 14051-14056 (1998), which is hereby incorporated by reference in its entirety). In particular, the growth and decay in whole-cell current initiated by carbamoylcholine in various conditions was described. Growth resulted from a stimulus at the acetylcholine-binding site and the decay includes receptor desensitization. The lines showing plateaus are integrated forms of these curves, permitting standardized evaluations of the maximum currents and times above baseline achieved in various conditions. Condition A (FIG. 4A) is a control experiment. Condition B (FIG. 4B) involved an aptamer with no effect on unimpaired carbamoylcholine. Condition C (FIG. 4C) is the same as condition A (FIG. 4A), but with the Class 1 compound cocaine present. Condition D (FIG. 4D) is the same as condition A (FIG. 4A), but with a Class 1 aptamer present. A combination of the cell-flow (Udgaonkar et al., “Chemical Kinetic Measurements of a Mammalian Acetylcholine Receptor by a Fast-Reaction Technique,” Proc. Natl. Acad. Sci. USA 84:8758-8762 (1987), which is hereby incorporated by reference in its entirety) and whole-cell current-recording (Hamill et al., “Improved Patch-Clamp Techniques for High-Resolution Current Recording From Cells and Cell-Free Membrane Patches,” Pflugers Arch. 391:85-100 (1981), which is hereby incorporated by reference in its entirety) techniques was used to record whole-cell currents at a membrane potential of −60 mV, 22° C. in BC₃H1 buffer, pH 7.4. Each cell was preincubated for 2 seconds with (A) 0.1 μg/μL tRNA and 0.3 unit/μL anti-RNase alone, (B) 5 μM Class 2 aptamer 3 plus 0.1 μg/μL tRNA and 0.3 unit/μL anti-RNase plus, (C) 100 μM cocaine plus 0.1 μg/μL tRNA and 0.3 unit/μL anti-RNase, or (D) 0.5 μM Class 1 aptamer 14 plus 0.1 μg/μL tRNA and 0.3 unit/μL anti-RNase. The whole-cell currents were then generated by 100 μM carbamoylcholine in the maintained presence of the compounds indicated. The lines parallel to the abscissa represents currents corrected for receptor desensitization (Udgaonkar et al., “Chemical Kinetic Measurements of a Mammalian Acetylcholine Receptor by a Fast-Reaction Technique,” Proc. Natl. Acad. Sci. USA 84:8758-8762 (1987), which is hereby incorporated by reference in its entirety).

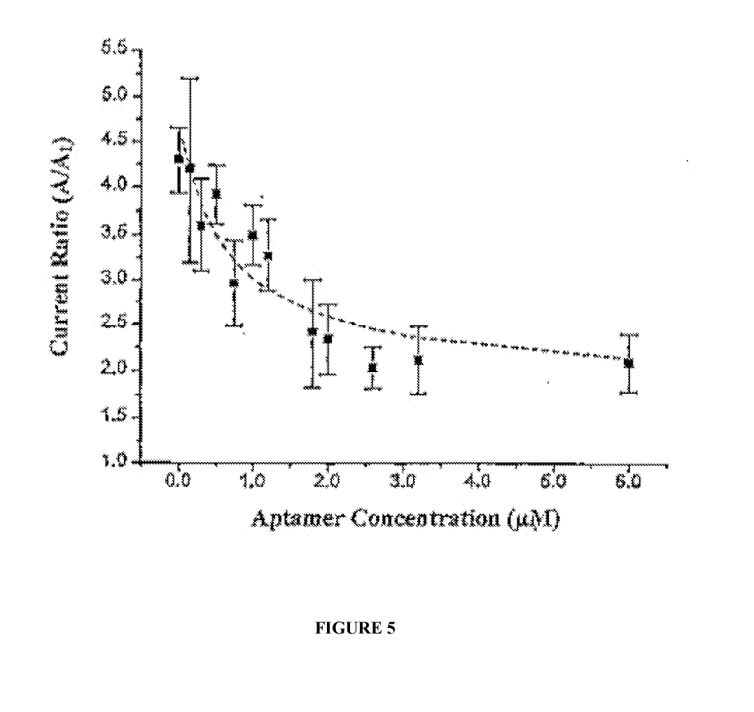

The effect of a Class 2 aptamer on the effect of the Class 1 compound cocaine is shown in FIG. 5, illustrating that the Class 2 aptamer alleviates, or reverses, the effect of cocaine in vitro (Hess et al., “Mechanism-Based Discovery of Ligands that Counteract Inhibition of the Nicotinic Acetylcholine Receptor by Cocaine and MK-801,” Proc. Nat. Acad. Sci. 97(25): 13895-13900 (2000), which is hereby incorporated by reference in its entirety). In FIG. 5, the presence of a Class 2 aptamer restores the carbamoylcholine response impaired in the condition C shown in FIG. 4C and discussed in the commentary on that figure (see supra). The baseline condition is the control carbamoylcholine response (1.0 on the y-axis). The concentration-dependent restoration of the carbamoylcholine response is shown as the concave line with maximum alleviation at the highest concentration of the Class 2 compound at the right-hand end of the x-axis. The y-axis shows a ratio of currents. Note that the symbols used for the y-axis have varied in different publications, using the symbol A or Amp for current, and subscripts (none, 0 or I) for baseline, inhibited and restored currents.

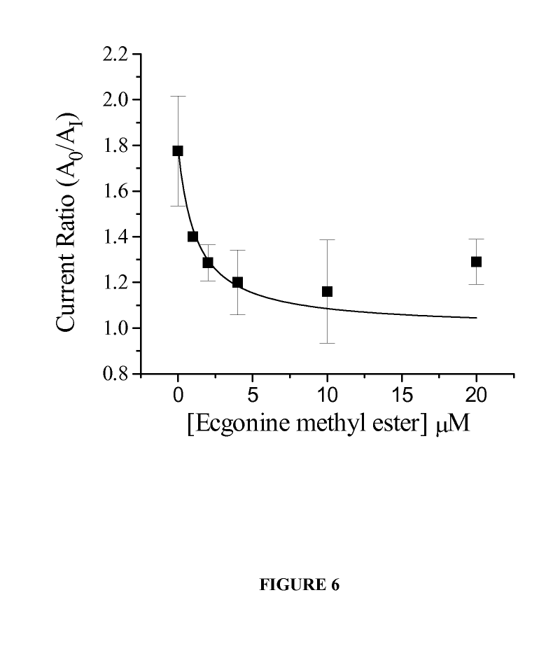

Alleviation of cocaine inhibition of the nicotinic acetylcholine receptor by the ligand EME in vitro is depicted in FIG. 6. At a constant concentration (100 μM) of carbamoylcholine, the ratio of the maximum current amplitudes obtained in the absence, A₀, and presence, A_{I}, of a constant concentration (200 μM) of cocaine was determined as a function of EME concentration. The cells were preincubated with 200-μM cocaine for 50 ms before a solution of carbamoylcholine with or without the other ligands, flowed over the cell. (Chen et al., “Mechanism-Based Discovery of Small Molecules that Prevent Noncompetitive Inhibition by Cocaine and MK-801 Mediated by Two Different Sites on the Nicotinic Acetylcholine Receptor,” Biochemistry 43:10149-10156 (2004), which is hereby incorporated by reference in its entirety). The prior studies, although in vitro, are consistent with the in vivo data in the data of the present invention.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of preventing and/or treating drug poisoning or drug addiction in a subject, said method comprising:
selecting a subject having or at risk of having drug poisoning or a drug addiction and
administering to the subject a ligand that binds to a regulatory site on nicotinic acetylcholine receptors under conditions effective to treat or prevent drug poisoning or drug addiction in the subject.

2. The method of claim 1 wherein said ligand has the following moiety:
wherein 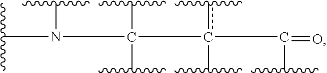 [symbol]
can be a single or a double bond; and 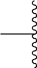
is the point of attachment of the moiety to the ligand.

3. The method according to claim 1, wherein said ligand comprises tropane or a derivative thereof having one of the following structures: 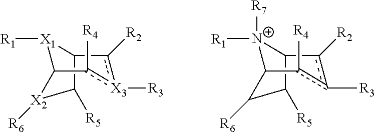
wherein
R₁, R₂, R₃, R₄, R₅, R₆, and R₇ are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁, X₂, and X₃ are independently selected from the group consisting of N, S, O, and C.

4. The method of claim 3, wherein said ligand is selected from the group consisting of ecgonine, ecgonine methyl ester, RTI-4229-70, RCS-III-143, RCS-III-140A, RCS-III-218, and RCS-III-202A.

5. The method according to claim 1, wherein said ligand comprises a cocaine analog selected from the group consisting of 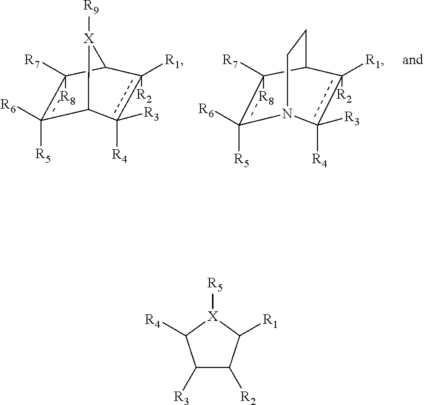
wherein
R₁, R₂, R₃, R₄, R₅, R₆, R₇, R₈, and R₉ are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and
X is independently selected from the group consisting of N, S, O, and C.

6. The method according to claim 1, wherein said ligand comprises piperidine or a derivative thereof having the structure 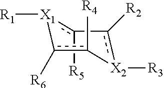
wherein
R₁, R₂, R₃, R₄, R₅, and R₆ are the same or different and are independently selected from the group consisting of hydrogen, hydroxyl, alkyl, cycloalkyl, alkenyl, alkoxy, aryl, alkylaryl, isoxazole, thiophene, indol, naphthalene, heterocyclic ring, halogen, and amine, as well as their esters and ethers, and X₁ and X₂ are independently selected from the group consisting of N, S, O, and C.

7. The method according to claim 1, wherein said ligand comprises a structure selected from the group consisting of 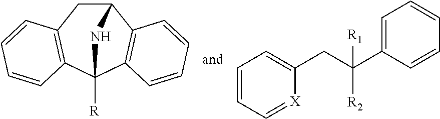
wherein
R, R₁, and R₂ are the same or different and are independently selected from the group consisting of hydrogen, hydroxyl, alkyl, cycloalkyl, alkenyl, alkoxy, aryl, alkylaryl, halogen, and amine, as well as their esters and ethers, and X is N or C.

8. The method according to claim 1, wherein said ligand comprises: 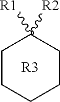
wherein

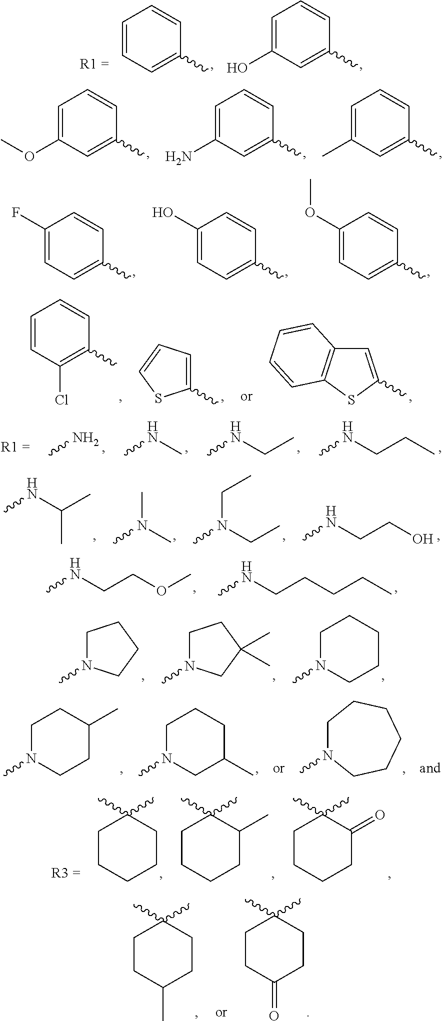

9. The method of claim 1, wherein said ligand that binds to a regulatory site on nicotinic acetylcholine receptors comprises an RNA aptamer.

10. The method of claim 9, wherein said RNA aptamer comprises a consensus sequence selected from the group of nucleotide sequences consisting of SEQ ID NO:1, SEQ ID NO:2, SEQ ID NO:3, SEQ ID NO:4, and SEQ ID NO:5.

11. The method of claim 9, wherein said RNA aptamer comprises a consensus sequence selected from the group of nucleotide sequences consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

12. The method according to claim 9, wherein said RNA aptamer comprises a nucleotide sequence selected from the group consisting of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

13. The method according to claim 9, wherein said RNA aptamer comprises a nucleotide sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, and SEQ ID NO:54.

14. The method of claim 9, wherein said RNA aptamer comprises a consensus sequence comprising a nucleotide sequence of SEQ ID NO:55.

15. The method according to claim 14, wherein said RNA aptamer comprises a nucleotide sequence selected from the group consisting of SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, and SEQ ID NO:65.

16. The method of claim 9, wherein said RNA aptamer comprises a consensus sequence comprising a nucleotide sequence of SEQ ID NO:66.

17. The method of claim 16, wherein said RNA aptamer comprises a nucleotide sequence selected from the group consisting of SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, and SEQ ID NO:87.

18. The method of claim 9, wherein said RNA aptamer comprises a consensus sequence comprising a nucleotide sequence of SEQ ID NO:88.

19. The method according to claim 18, wherein said RNA aptamer comprises a nucleotide sequence selected from the group consisting of SEQ ID NO:89, SEQ ID NO:90, SEQ ID NO:91, SEQ ID NO:92, SEQ ID NO:93, SEQ ID NO:94, and SEQ ID NO:95.

20. The method of claim 9, wherein said RNA aptamer is chemically modified.

21. The method according to claim 20, wherein said chemically modified RNA aptamer comprises one or more modified nucleotides.

22. The method according to claim 1, wherein said administering is carried out orally, parenterally, nasally, subcutaneously, intravenously, intramuscularly, intracerebroventricularly, intraparenchymal, intraperitoneally, by intranasal inhalation, by implantation, by intracavitary or intravesical instillation, intraocularly, intraarterially, intralesionally, transdermally, or by application to mucous membranes.

23. The method according to claim 1, wherein the subject is a human.

24. The method of claim 1, wherein the drug poisoning or drug addiction is treated in the selected subject.

25. The method of claim 1, wherein the drug poisoning or drug addiction is prevented in the selected subject.

26. The method of claim 1, wherein the drug poisoning or drug addiction involves one or more drugs selected from the group consisting of phencyclidine (PCP), marijuana, cocaine, and nicotine.
